(19) United States
(12) Patent Application Publication
(10) Pub. No.: US 2019/0365251 A1
(43) Pub. Date: Dec. 5, 2019

(54) BIOLOGICAL INFORMATION MEASURING APPARATUS AND METHOD AND PROGRAM USING THE SAME

(71) Applicants: OMRON CORPORATION, Kyoto (JP); OMRON HEALTHCARE CO., LTD., Kyoto (JP)

(72) Inventors: Tsuyoshi KITAGAWA, Kyoto (JP); Shingo YAMASHITA, Kyoto (JP); Katsunori KONDO, Kyoto (JP); Yuki KATO, Kyoto (JP)

(21) Appl. No.: 16/544,986

(22) Filed: Aug. 20, 2019

Related U.S. Application Data
(63) Continuation of application No. PCT/JP2018/009560, filed on Mar. 12, 2018.

(30) Foreign Application Priority Data
Mar. 15, 2017 (JP) 2017-050562

(51) Int. Cl. A61B 5/0225; A61B 5/021; A61B 5/0235; A61B 5/00

(52) U.S. Cl. CPC A61B 5/0225; A61B 5/02108; A61B 5/02141; A61B 2562/0247; A61B 5/7445; A61B 5/6824; A61B 2560/0223; A61B 5/0235

(57) ABSTRACT

According to one embodiment, a biological information measuring apparatus includes a detector, a measuring device, and a connector. The detector detects a pulse wave in a temporally continuous manner. The measuring device intermittently measures biological information and calibrate the pulse wave with the biological information. The connector having impact absorbability physically connects and integrates the detector and the measuring device.

### CROSS-REFERENCE TO RELATED APPLICATIONS

This application is a Continuation Application of PCT Application No. PCT/JP2018/009560, filed Mar. 12, 2018 and based upon and claiming the benefit of priority from Japanese Patent Application No. 2017-050562, filed Mar. 15, 2017, the entire contents of all of which are incorporated herein by reference.

### FIELD

The present invention relates generally to a biological information measuring apparatus, and a method and a program using the same, for continuously measuring biological information.

### BACKGROUND

With the development of sensor technology, since an environment where high-performance sensors can be easily used has been established, the importance of utilizing biological information to detect biological abnormalities early on and using it for treatment has been gradually increasing in medical treatment.

A biological information measuring apparatus is known, which is capable of measuring biological Information of such as pulse and blood pressure using information detected by the pressure sensor in a state where the pressure sensor is in direct contact with a living body site through which an artery such as the radial artery of the wrist passes (see, for example, Jpn. Pat. Appln. KOKAI Publication No. 2004-113368).

The blood pressure measuring apparatus described in Jpn. Pat. Appln. KOKAI Publication No. 2004-113368 calculates a blood pressure value using a cuff at a site different from the living body site to which the pressure sensor is made to contact, and generates calibration data from the calculated blood pressure value. The blood pressure value is calculated for each beat by calibrating the pressure pulse wave detected by the pressure sensor using this calibration data.

However, in the blood pressure measuring apparatus described in Jpn. Pat. Appln. KOKAI Publication No. 2004-113368, a plurality of devices are required. Furthermore, since the apparatus is large, it is difficult to increase the measurement accuracy. In addition, since the apparatus is premised on being used in a limited environment, and being operated by a specific person, it is difficult to be used for daily medical care and at home. Furthermore, since this blood pressure measuring apparatus has many tubes and wires, which are troublesome, it is not practical to be used daily or during sleep.

### SUMMARY

A first aspect of the present invention comprises a detector that detects pulse waves in a temporally continuous manner, a measuring device that measures biological information intermittently and calibrates the pulse waves based on the biological information, and a connecting unit having impact absorbability that physically connects and integrates the detector and the measuring device.

A second aspect of the present invention comprises a detector that detects pulse waves in a temporally continuous manner, a measuring device that measures biological information intermittently and calibrates the pulse waves based on the biological information, and a connecting unit that physically connects the detector and the measuring device.

According to a third aspect of the present invention, the detector is smaller in capacity and mass than the measuring device.

A fourth aspect of the present invention further comprises a drive unit that drives a pressing unit included in the detector and a cuff included in the measuring device, and an electric power source unit that supplies power to devices included in the detector and the measuring device. The drive unit and the power source are included in the measuring device.

According to a fifth aspect of the present invention, the drive unit includes a pump-and-valve and a pressure sensor, and adjusts the pressure of the cuff or the pressing unit.

A sixth aspect of the present invention further comprises a first drive unit that drives a pressing unit included in the detector, a second drive unit that drives a cuff included in the measuring device, and an electric power source unit that supplies power source to devices included in the detector and the measuring device. The first drive unit is included in the detector, and the second drive unit and the electric power source unit are included in the measuring device.

According to a seventh aspect of the present invention, each of the first drive unit and the second drive unit includes a pump-and-valve and a pressure sensor, and adjusts the pressure of the cuff or the pressing unit.

An eighth aspect of the present invention further comprises a display that displays a detection result of the detector or a measurement result of the measuring device.

The display is included in the measuring device.

A ninth aspect of the present invention further comprises a first display that displays a detection result of the detector, and a second display that displays a measurement result of the measuring device. The first display is included in the detector, and the second display is included in the measuring device.

A tenth aspect of the present invention further comprises an operation unit that operates the detector and the measuring device. The operation unit is included in the measuring device.

An eleventh aspect of the present invention further comprises a first operation unit that operates the detector, and a second operation unit that operates the measuring device. The first operation unit is included in the detector, and the second operation unit is included in the measuring device.

According to a twelfth aspect of the present invention, the connecting unit extends in a direction connecting the detector and the measuring device in a straight line, and connects the detector and the measuring device.

According to a thirteenth aspect of the present invention, the connecting unit extends in a direction intersecting the direction connecting the detector and the measuring device in a straight line, and connects the detector and the measuring device.

According to a fourteenth aspect of the present invention, the detector and the measuring device are installed on a wrist, and the connecting unit extends from the detector and the measuring device in a direction intersecting a direction in which an arm is extended to connect the detector and the measuring device.

According to a fifteenth aspect of the present invention, the connecting unit connects the detector and the measuring device with a detachable connector.

According to a sixteenth aspect of the present invention, a part of the connector is connected to a signal line that transmits an electrical signal between the detector and the measuring device, and, in a case where the drive unit is included only in the measuring device, another part of the connector is connected to a tube through which gas flows in and out between the detector and the measuring device.

According to a seventeenth aspect of the present invention, the connecting unit connects the detector and the measuring device with a tube having a bellows structure.

According to an eighteenth aspect of the present invention, the connecting unit connects the detector and the measuring device by a universal joint.

According to a nineteenth aspect of the present invention, the measuring device measures biological information more accurately than biological information obtained from the detector.

According to a twentieth aspect of the present invention, the detector detects the pulse wave for each pulse. The biological information is blood pressure.

According to the first aspect of the present invention, since the detector that detects pulse waves in a temporally continuous manner, and the measuring device that intermittently measures biological information are physically connected and integrated, the biological information measuring apparatus is made compact. Therefore, the biological information measuring apparatus can be easily worn and perform measurement, which is highly convenient for a user. Also, since the measuring device only performs intermittent measurement, the time at which the measuring device interferes with the user is reduced. Furthermore, since the connecting unit has impact absorbability, even when the measuring device operates to measure biological information, vibration and impact are absorbed by the connecting unit and are hardly transmitted to the detector. As a result, the accuracy of pulse wave measurement in the detector is improved.

According to the second aspect of the present invention, by comprising a detector that detects pulse waves in a temporally continuous manner, a measuring device that intermittently measures biological information and calibrates the pulse waves based on biological information, and the connecting unit that physically connects the detector and the measuring device, the biological information measuring apparatus can integrate the detector and the measuring device. As a result, since the biological information measuring apparatus becomes compact, it can be easily worn and highly convenient for the user. In addition, by connecting the detector and the measuring device by the connecting unit, the vibration and impact of the detector and the measuring device, can be absorbed, thereby, improving detection accuracy and measurement accuracy of the detector and the measuring device in comparison to the case in which the connecting unit is not provided. Furthermore, the biological information measuring apparatus can be made more compact by bringing the detector and the measuring device close to each other by the arrangement of the connecting unit.

According to the third aspect of the present invention, since the detector has a smaller capacity and mass than the measuring device, the detector can be easily installed at a desired position. As a result, the detector can reliably detect the pulse wave, and the accuracy in detecting the pulse wave of the detector is increased.

According to the fourth aspect of the present invention, since the drive unit and the electric power source unit are included in the measuring device, the detector is made compact and lightweight, and can be easily installed at a desired position and reliably acquire a pulse wave. As a result, the accuracy of pulse wave measurement of the detector is improved.

According to the fifth aspect of the present invention, since the drive unit includes the pump-and-valve and the pressure sensor, and is included in the measuring device, but not in the detector, the detector can be made as a device that does not have a large capacity and mass for handling gas. As a result, the capacity and mass of the detector are relatively reduced, and the detector can be easily installed at a desired position and reliably acquire a pulse wave.

The sixth aspect of the present invention further comprises the first drive unit that drives the pressing unit included in the detector, and the second drive unit that drives the cuff included in the measuring device. Since the drive unit is provided in each of the detector and the measuring device, there is no need to pass a tube between the detector and the measuring device for passing the gas through and adjusting the pressure.

According to the seventh aspect of the present invention, since the first drive unit and the second drive unit respectively include the pump-and-valve and the pressure sensor, the pump-and-valve can be controlled independently. In addition, a tube for moving the gas to the connecting unit becomes unnecessary, and, in the case where the connecting unit moves, a force is hardly applied to the tube. As a result, the tube connecting the pump and the valve to move gas is less likely to break.

According to the eighth aspect of the present invention, since the display for displaying the detection result of the detector or the measurement result of the measuring device is only present in the measuring device, the detector can be compact and lightweight, can be easily installed at a desired position, and can reliably acquire the pulse wave. As a result, the accuracy of pulse wave measurement of the detector is improved.

According to the ninth aspect of the present invention, by installing the display unit to each of the detector and the measuring device, different contents can be displayed respectively therein. For example, the detector displays the measured blood pressure value in real time, and the measuring device displays the blood pressure value at the time of the previous calibration or displays the current capacity of the power source. As a result, the user can obtain much information from the display.

According to the tenth aspect of the present invention, by providing the operation unit only in the measuring device, the detector can be made compact. As a result, the detector can be easily installed at a desired position, and the detector can reliably acquire a pulse wave. As a result, the accuracy of pulse wave measurement of the detector is improved.

According to the eleventh aspect of the present invention, by installing the operation unit on each of the detector and the measuring device, an operation unit including operations specific to each of the detector and the measuring device can be installed, thereby, improving the convenience for the user.

According to the twelfth aspect of the present invention, since the connecting unit is disposed in the direction connecting the detector and the measuring device in a straight line, the connecting unit can absorb vibration and impact of the detector and the measuring device. As a result, the detection accuracy and the measurement accuracy of the detector and the measuring device are improved compared to the case where there is no connecting unit.

According to the thirteenth aspect of the present invention, since the connecting unit is disposed in the direction intersecting the direction connecting the detector and the measuring device by a straight line, the detector and the measuring device can be brought close to each other. As a result, the biological information measuring apparatus can be made more compact.

According to the fourteenth aspect of the present invention, the detector and the measuring device are installed on the wrist, and the connecting unit extends from the detector and the measuring device in the direction intersecting the direction in which the arm is extended, so that the detector and the measuring device can be brought close to each other. According to this aspect, since a gap can be provided between the connecting unit and the measuring device, the connecting unit can absorb the vibration and impact of the detector and the measuring device. Furthermore, since the connecting unit extends from the detector and the measuring device in the direction intersecting the direction in which the arm extends, the detector and the measuring device can be freely disposed depending on the length of the connecting unit in the arm direction. As a result, since the detector and the measuring device can be easily disposed at desired positions, the detector can reliably acquire a pulse wave, and the measuring device can measure biological information with high accuracy.

According to the fifteenth aspect of the present invention, since the connecting unit is connected to the detector and the measuring device by a detachable connector. the detector and the measuring device can be separated. Thus, when one of the devices fails, only the failed device needs to be replaced. Therefore, it is convenient for the user since only the failed device needs to be replaced.

According to the sixteenth aspect of the present invention, a part of the connector is connected to a signal line for transmitting an electrical signal between the detector and the measuring device, and the other part of the connector is connected to the tube through which gas flows in and out between the detector and the measuring device, so that the detector and the measuring device can be separated, and both signal lines and tubes of the connecting unit are connected to the connector. Therefore, even if the detector or the measuring device is replaced, the signal line and the tube can be used in the exact same manner as before replacement, which is convenient for the user.

According to the seventeenth aspect of the present invention, the tube having the bellows structure allows the arrangement of the detector and the measuring device to be freely changed, and positioning is possible not only in the direction of expansion and contraction but also in the direction perpendicular to this direction. As a result, the detector and the measuring device do not easily interfere with each other. As a result, the measurement accuracy of the detector and the measuring device is improved.

According to the eighteenth aspect of the present invention, by connecting the measuring device and the detector by the universal joint, the arrangement of the detector and the measuring device can be freely changed, and the detector and the measuring device are less likely to interfere with each other. Therefore, the measurement accuracy of the detector and the measuring device is improved.

According to the nineteenth aspect of the present invention, the measuring device measures the biological information with higher accuracy than the biological information obtained from the detector. Therefore, highly accurate biological information obtained from the measuring device is calibrated. Since this ensures the accuracy of biological information obtained based on a pulse wave from the detector, it becomes possible to calculate biological information with high accuracy in a temporally continuous manner.

According to the twentieth aspect of the present invention, the detector detects a pulse wave for each pulse. Since the biological information is blood pressure, the biological information measuring apparatus can measure blood pressure for each pulse wave in a temporally continuous manner.

That is, according to each aspect of the present invention, it is possible to provide a biological information measuring apparatus, and a method and a program using the same, in which the biological information measuring apparatus is capable of acquiring accurate information while being constantly worn to calibrate biological information in a temporally continuous manner.

### BRIEF DESCRIPTION OF THE DRAWINGS

FIG. 1 is a block diagram showing a blood pressure measuring apparatus according to an embodiment.

FIG. 2 shows an example in which the blood pressure measuring apparatus of FIG. 1 is attached to a wrist.

FIG. 3 shows another example in which the blood pressure measuring apparatus of FIG. 1 is attached to the wrist.

FIG. 4 shows a time course of a cuff pressure and a pulse wave signal in an oscillometric method.

FIG. 5 shows a time change of pulse pressure per pulse and one of the pulse waves.

FIG. 6 is a flowchart showing a first calibration method.

FIG. 7A shows an example of the connecting unit of FIG. 1 being made of an impact absorbing material.

FIG. 7B shows another example of the connecting unit of FIG. 1 being made of an impact absorbing material.

FIG. 7C is a diagram in which a signal line and a duct run through a tube of the connecting unit of FIG. 1.

FIG. 8A shows an example in which a connecting line is outside the apparatus and is connected in FIG. 7A.

FIG. 8B shows an example in which the connecting unit is outside the apparatus and is connected.

FIG. 9A shows an example in which the connecting unit of FIG. 1 includes air.

FIG. 9B shows another example in which the connecting unit of FIG. 1 includes air.

FIG. 10A shows an example in which the connecting unit of FIG. 1 is made of an impact absorbing material and includes a connector for connecting a signal line and a tube.

FIG. 10B shows another example in which the connecting unit of FIG. 1 is made of an impact absorbing material and includes a connector for connecting a signal line and a tube.

FIG. 11A shows an example in which the connecting unit of FIG. 1 has a bellows structure.

FIG. 11B shows an example in which the connecting unit of FIG. 1 is a universal joint.

### DETAILED DESCRIPTION

Hereinafter, a biological information measuring apparatus, and a method and a program using the same, according to embodiments of the present invention will be explained with reference to the drawings. In the following embodiments, the portions given the same numbers perform the same operations. There, explanations thereof will be omitted.

The present embodiments have been made in view of the above circumstances, and its object is to provide a biological information measuring apparatus, and a method and a program using the same, in which the biological information measuring apparatus is capable of acquiring accurate information while being constantly worn to calibrate biological information in a temporally continuous manner.

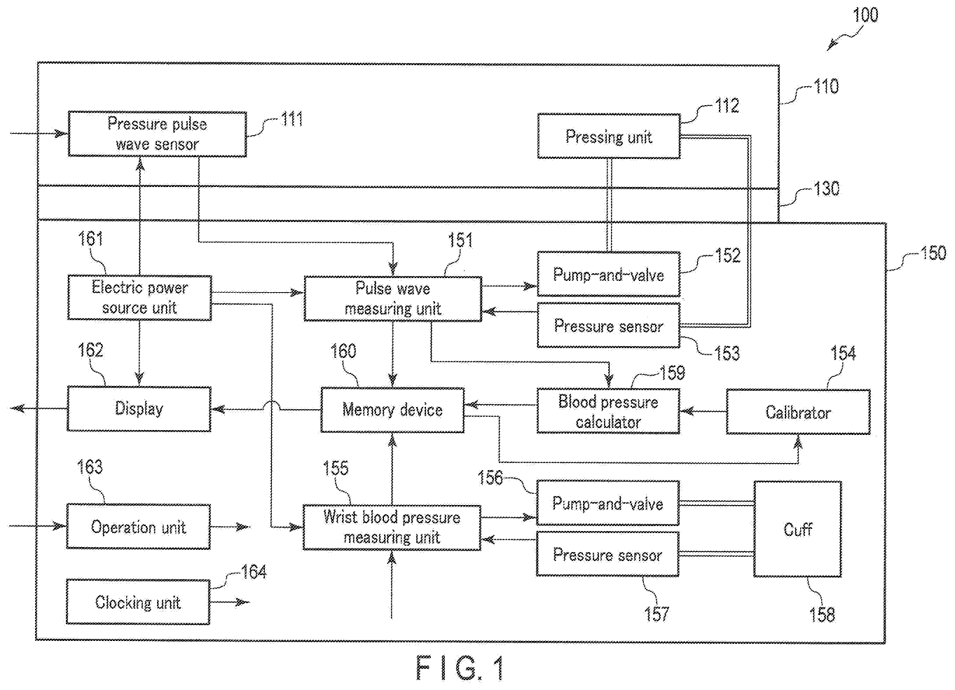

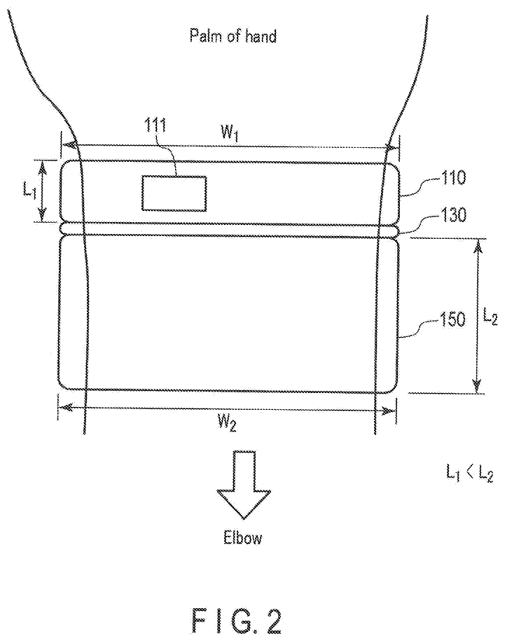

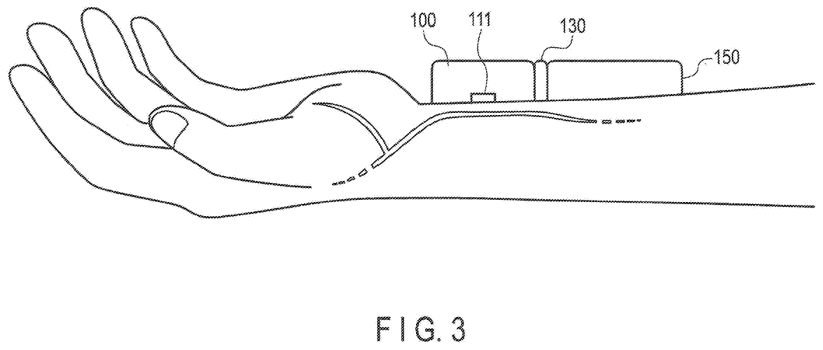

An example of a blood pressure measuring apparatus IOC of a biological information measuring apparatus according to the present embodiment will be explained with reference to FIG. 1, FIG. 2 and FIG. 3. FIG. 1 is a functional block diagram of the blood pressure measuring apparatus 100, which shows details of a pulse wave detector 110 and a blood pressure measuring device 150. PIG. 2 shows an example in which the blood pressure measuring apparatus 100 is attached to the wrist, and is a schematic perspective view seen from above the palm. A pressure pulse wave sensor 111 is arranged on a side closer to the wrist on the pulse wave detector 110. FIG. 3 is an image diagram of the blood pressure measuring apparatus 100 being worn, and is a schematic perspective view where the palm is viewed from the side (the direction in which the fingers line up when opening the hand). FIG. 3 shows an example in which the pressure pulse wave sensor 111 is arranged orthogonal to the radial artery. In FIG. 3, the blood pressure measuring apparatus 100 may seem to be simply placed on the palm side of the arm. In reality, however, the blood pressure measuring apparatus 100 is wound around the arm.

The blood pressure measuring apparatus 100 includes a pulse wave detector 110, a connecting unit 130, and a blood pressure measuring device 150. The pulse wave detector 110 includes a pressure pulse wave sensor 111 and a pressing unit 112. The blood pressure measuring device 150 includes a pulse wave measuring unit 151, a pump-and-valve 152, a pressure sensor 153, a calibrator 154, a wrist blood pressure measuring unit 155, a pump-and-valve 156, a pressure sensor 157, a cuff 158, a blood pressure calculator 159, a memory device 160, an electric power source unit 161, a display 162, an operation unit 163, and a clocking unit 164.

The blood pressure measuring apparatus 100 forms an annular shape and measures blood pressure by being wound like a bracelet around the wrist, etc. As shown in FIG. 2 and FIG. 3, the pulse wave detector 110 is arranged on a side of the wrist which is closer to the palm than the blood pressure measuring device 150. In other words, the pulse wave detector 110 is disposed at a position farther from the elbow than the blood pressure measuring device 150. In the present embodiment, the pulse wave detector 110 is arranged in a manner that the pressure pulse wave sensor 111 is located on the radial artery. This arrangement brings the blood pressure measuring device 150 closer to the elbow than the pulse wave detector 110. The connecting unit 130 physically connects the pulse wave detector 1.10 and the blood pressure measuring device 150, and is made of, for example, an impact absorbing material, so that the pulse wave detector 110 and the blood pressure measuring device 150 do not interference with each other's measurements.

A length L₁ of the pulse wave detector 110 in the extending direction of the arm is set smaller than a length L₂ of the blood pressure measuring device 150 in the extending direction. The length Li of the pulse wave detector 110 in the extending direction of the arm is set to 40 mm or less, and more ideally to 15 to 25 mm. In addition, a length W₁ of the pulse wave detector 110 in a perpendicular direction to the extending direction of the arm is set to 4 to 5 cm, and a length W₂ of the blood pressure measuring device 150 in a perpendicular direction to the extending direction is set to 6 to 7 cm. In addition, the length Wi and the length W₂ have a relationship of 0 (or 0.5) cm<W₂−W₁<2 cm. This relationship prevents W₂ from being set too long, and makes it difficult to interfere with the surroundings. By keeping the pulse wave detector 110 within such width, the blood pressure measuring device 150 is disposed closer to the palm side, so that the pulse wave can be easily detected, and measurement accuracy can be maintained.

The pressure pulse wave sensor 111 detects the pressure pulse wave in a temporally continuous manner. For example, the pressure pulse wave sensor 111 detects a pressure pulse wave for each pulse. The pressure pulse wave sensor 111 is disposed on the palm side as shown in FIG. 2, and is generally disposed parallel to the extending direction of the arm as shown in FIG. 3. The pressure pulse wave sensor 111 can obtain time-series data of a blood pressure value (blood pressure waveform) that changes in conjunction with the heartbeat.

By acquiring from the clocking unit 164, a time when the pulse wave measuring unit 151 received a pressure pulse wave from the pressure wave sensor 111, it is possible to estimate a time when the pressure pulse wave sensor 111 detected the pressure pulse wave.

The pressing unit 112 is an air bag and can increase the sensor sensitivity by pressing the sensor portion of the pressure pulse wave sensor 111 against the wrist.

The pulse wave measuring unit 151 receives from the pressure pulse wave sensor 111, pressure pulse wave data along with a time, and transmits this data to the memory device 160 and the blood pressure calculator 159. The pulse wave measuring unit 151 pressurizes or depressurizes the pressing unit 112 by driving and controlling the pump-and-valve 152 and the pressure sensor 153, and adjusts the pressure pulse wave sensor 111 in a manner to be pressed against the radial artery of the wrist.

The pump-and-valve 152 pressurize or depressurize the pressing unit 112 according to an instruction from the pulse wave measuring unit 151. The pressure sensor 153 monitors the pressure of the pressing unit 112 and notifies the pulse wave measuring unit 151 of a pressure valve of the pressing unit 112. Here, the pump-and-valve 152 and the pressure sensor 153 are installed only in the blood pressure measuring device 150; however, they may also be installed in the pulse wave detector 110 together with units that drive and control the pump-and-valve 152 and the pressure, sensor 153. In this case, it is unnecessary to pass a tube for adjusting the pressure through the gas between the pulse wave detector 110 and the blood pressure measuring device 150.

The wrist blood pressure measuring unit 155 measures blood pressure, which is the biological information, with higher accuracy than the pressure pulse wave sensor 111. For example, the wrist blood pressure measuring unit 155 measures the blood pressure intermittently, not in a temporally continuous manner, and transmits the measured value to the calibrator 154. The wrist blood pressure measuring unit 155 measures the blood pressure using, for example, the oscillometric method. The pulse wave measuring device 155 pressurizes or depressurizes the cuff 158 by controlling the pump-and-valve 156 and the pressure sensor 157, and measures the blood pressure. The wrist blood pressure measuring unit 155 transmits to the memory device 160 the systolic blood pressure together with a time when this systolic blood pressure was measured, and the diastolic blood pressure together with a time when this diastolic blood pressure was measured. The systolic blood pressure is also referred to as SBP, and the diastolic blood pressure is also referred to as DBP.

The memory device 160 sequentially acquires from the pulse wave measuring unit 151, the pressure pulse wave data together with a detection time, and stores them. The memory device 160 also acquires from the wrist blood pressure measuring unit 155, a SBP measurement time together with the SBP, and a DBP measurement time together with the DBP that, are acquired when this measuring unit operates.

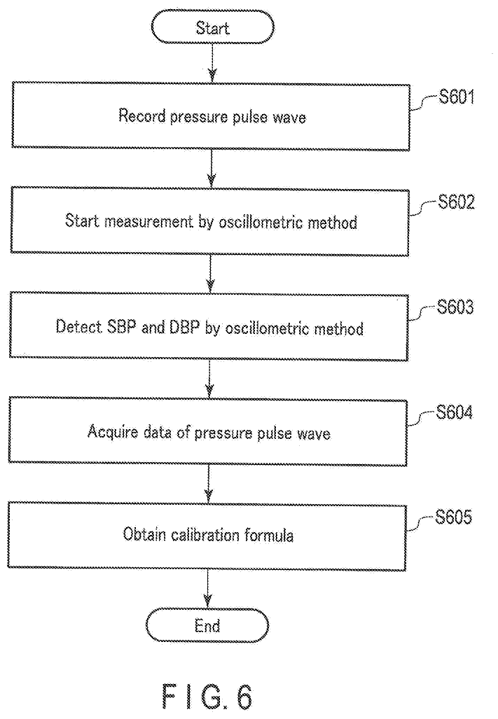

The calibrator 154 acquires from the memory device 160, the SBP and DBP measured by the wrist blood pressure measuring unit 155 together with the measurement times, and the pressure pulse wave data measured by the pulse wave measuring unit 151 together with the measurement time. The calibrator 154 calibrates the pressure pulse wave from the pulse wave measuring unit 151 based on the blood pressure value from the wrist blood pressure measuring unit 155. Although there are several possible calibration methods performed by the calibrator 154, the calibration method will be described in detail later on with reference to FIG. 6.

The blood pressure calculator 159 receives the calibration method from the calibrator 154, calibrates the pressure pulse wave data from the pulse wave measuring unit 151, and causes the memory device 160 to store the blood pressure data obtained from the calibrated pressure pulse wave data, together with the measurement time.

The electric power source unit 161 supplies power to each of the pulse wave detector 110 and the blood pressure measuring device 150.

The display 162 displays various types of information, such as a result of a blood pressure measurement, to the user. For example, the display 162 receives data from the memory device 160 and displays the contents of the data. For example, the display 162 displays pressure pulse wave data together with measurement time. Here, the display 162 is installed only in the blood pressure measuring device 150, however, it may be installed in the pulse wave detector 110. In this case, for example, the pulse wave detector 110 displays the measured blood pressure value in real time, and the blood pressure measuring device 150 displays the blood pressure value at the time of the previous calibration or displays the current capacity of the power source. As a result, the user can obtain much information from the display.

The operation unit 163 receives an operation from the user. The operation unit 163 includes, for example, an operation button for causing the wrist blood pressure measuring unit 155 to start measurement, and an operation button for performing calibration.

Here, although the operation unit 163 is installed only in the blood pressure measuring device 150, the operation unit 163 may be installed in the pulse wave detector 110.

The clocking unit 164 generates time and supplies it to a unit that is in need thereof. For example, the memory device 160 records time as well as the data to be stored.

At the time of implementation, a program for executing the above-described operations is stored in, for example, a secondary storage device included in each of the pulse wave measuring unit 151, the calibrator 154, the blood pressure calculator 159, and the wrist blood pressure measuring unit 155, and the central processing unit (CPU) executes a read operation of the stored program. The secondary storage device is, for example, a hard disk; however, it may be any device capable of storing data, such as a semiconductor memory, a magnetic memory device, an optical memory device, a magneto-optical disk, and a memory device employing the phase-change recording technology.

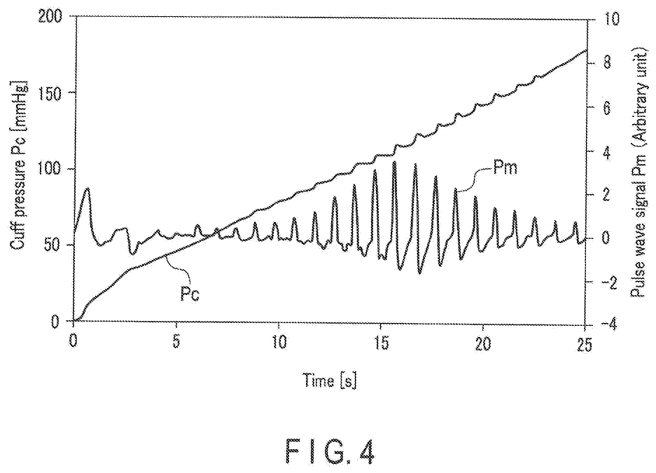

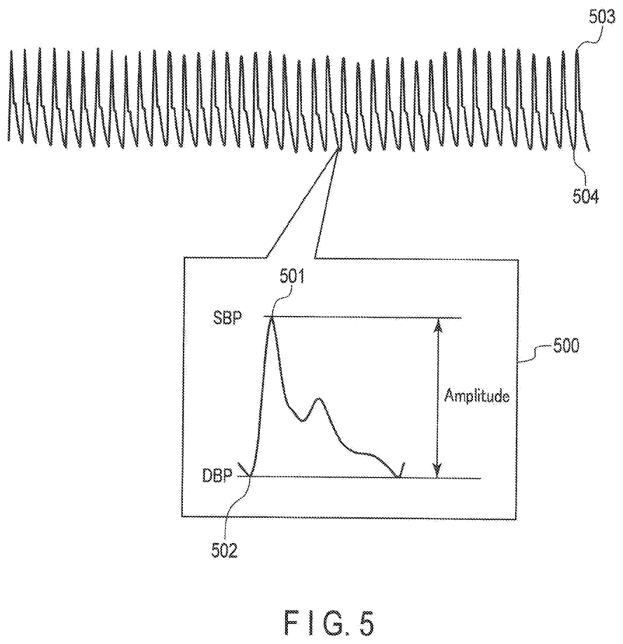

Next, with reference to FIG. 4 and FIG. 5, operations performed by the pulse wave measuring unit 151 and the wrist blood pressure measuring unit 155 prior to calibration by the calibrator 154 will be explained. FIG. 4 illustrates changes in cuff pressure and changes in magnitude of the pulse wave signal over time, during a blood pressure measurement by the oscillometric method. FIG. 4 illustrates changes in cuff pressure and changes in pulse wave signal over time, in which the cuff pressure increases with time, and the magnitude of the pulse wave signal gradually increases with the increase in the cuff pressure, and gradually decreases after reaching the maximum value. FIG. 5 shows time-series data of pulse pressure when pulse pressure per pulse is measured. FIG. 5 shows the waveform of one of the pressure pulse waves.

First, the operation of when the wrist blood pressure measuring unit 155 measures the blood pressure by the oscillometric method will be briefly explained with reference to FIG. 4. A blood pressure value may be calculated not only in a pressurizing process but also in a depressurizing process. However, only the pressurizing process will be explained herein.

When a user instructs blood pressure measurement by the oscillometric method via the operation unit 163 provided in the blood pressure measuring device 150, the wrist blood pressure measuring unit 155 starts operating and initializes the processing memory area. The wrist blood pressure measuring unit 155 also turns off the pump and opens the valve of the pump-and-valve 156 to exhaust the air inside the cuff 158. Subsequently, the wrist blood pressure measuring unit 155 performs control to set a present output value of the pressure sensor 157 as a value corresponding to the atmospheric pressure (0 mmKg adjustment).

Subsequently, the wrist blood pressure measuring unit 155 operates as a pressure controller to close the valve of the pump-and-valve 156, then drives the pump to control air to be delivered to the cuff 158. As a result, the cuff 158 is inflated, and the cuff pressure (Pc in FIG. 4) is gradually increased and pressurized. In this pressurizing process, the wrist blood pressure measuring unit 155 monitors the cuff pressure Pc via the pressure sensor 157 in order to calculate a blood pressure value, and acquires, as a pulse wave signal Pm as shown in FIG. 4, a fluctuation component of the arterial volume that is generated in the radial artery of the wrist, which is a site to be measured.

Then, based on the pulse wave signal Pm acquired at that point in time, the wrist blood pressure measuring unit 155 applies a known algorithm by the oscillometric method and attempts to calculate the blood pressure values (SBP and DBP). If the blood pressure value cannot be calculated at this point in time because of shortage of data, the pressurizing processing is repeated in the same manner as the above unless the cuff pressure Pc has reached the upper limit pressure (for safety, preset to, for example, 300 mmHg (to be exact, this value is a pressurizing value)).

When the blood pressure value is calculated in this manner, the wrist blood pressure measuring unit 155 stops the pump and opens the valve of the pump-and-valve 156 to perform control of exhausting the air inside the cuff 158. Finally, the measurement result of the blood pressure value is passed to the calibrator.

Next, the matter of the pulse wave measuring unit 151 measuring the pulse wave for each pulse will be explained with reference to FIG. 5. The pulse wave measuring unit 151 measures the pulse wave by, for example, the tonometry method.

The pulse wave measuring unit 151 controls the pump-and-value 152 and the pressure sensor 153 to provide an optimum pressing force that is determined in advance for the pressure pulse wave sensor 111 to realize an optimum measurement, and increases the internal pressure of the pressing unit 112 to the optimum pressing force, and maintains the optimum pressing force. Then, when the pressure pulse wave is detected by the pressure pulse wave sensor 111, the pulse wave measuring unit 151 acquires the pressure pulse wave.

The pressure pulse wave is detected for each pulse as a waveform as shown in FIG. 5, and respective pressure pulse waves are detected continuously. The pressure pulse wave 500 in FIG. 5 represents a pressure pulse wave of one pulse, in which the pressure value of 501 corresponds to SBP, and the pressure value of 502 corresponds to DBP. As shown in the pressure pulse wave time-series of FIG. 5, normally, SBP 503 and DBP 504 fluctuate for each pressure pulse wave.

Next, the operation of the calibrator 154 will be explained with reference to FIG. 6.

The calibrator 154 calibrates the pressure pulse wave detected by the pulse wave measuring unit 151 using the blood pressure value measured by the wrist blood pressure measuring unit 155. That is, the calibrator 154 determines the blood pressure values of the maximum value 501 and the minimum value 502 of the pressure pulse wave detected by the pulse wave measuring unit 151.

(Calibration Method)

The pulse wave measuring unit 151 starts recording the pressure pulse wave data of the pressure pulse wave, and sequentially stores the pressure pulse wave data in the memory device 160 (step S601). Thereafter, for example, a user activates the wrist blood pressure measuring unit 155 using the operation unit 163 to start measurement by the oscillometric method (step S602). Based on the pulse wave signal Pm, the wrist blood pressure measuring unit 155 records SBP data and DBP data obtained by detecting SBP and DBP by the oscillometric method, respectively, and stores the recorded SBP data and DBP data in the memory device 16G (step SS03).

The calibrator 154 acquires pressure pulse waves corresponding to the SBP data, and the DBP data from the pressure pulse wave data (step S604). The calibrator 154 obtains a calibration formula based on the maximum value 501 of the pressure pulse wave corresponding to SBP and the minimum value 502 of the pressure pulse wave corresponding to DBP (step S605).

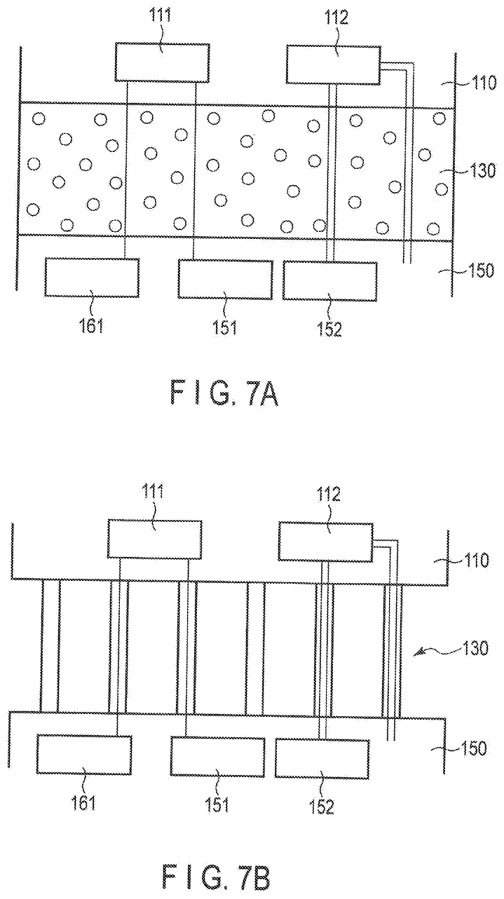

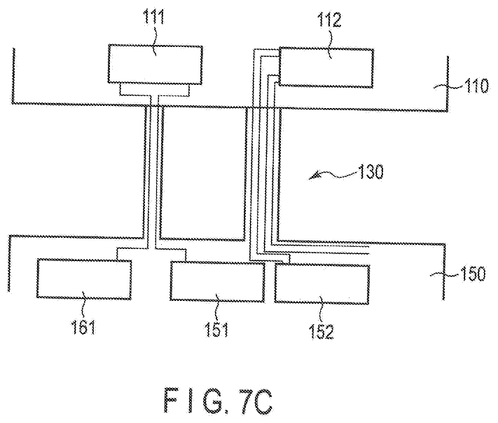

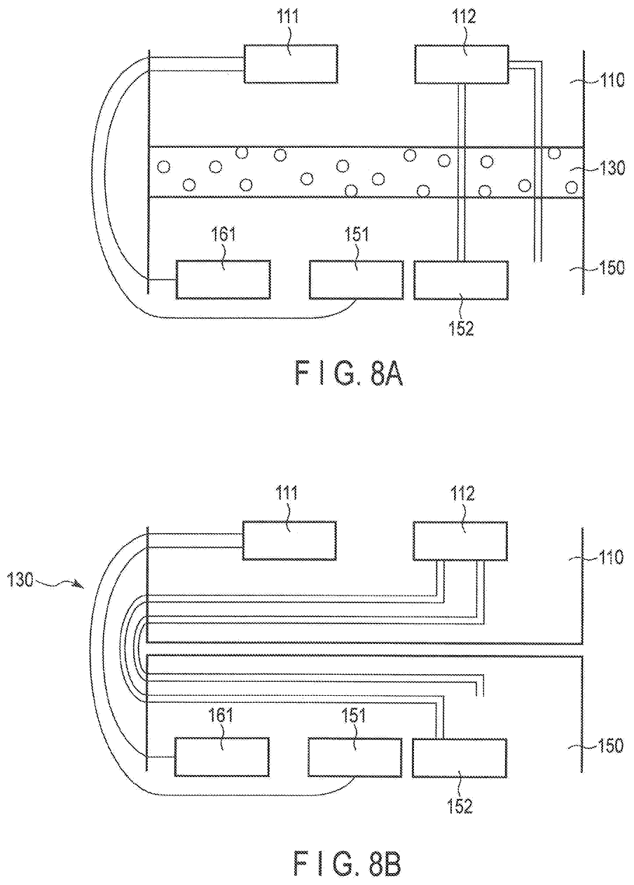

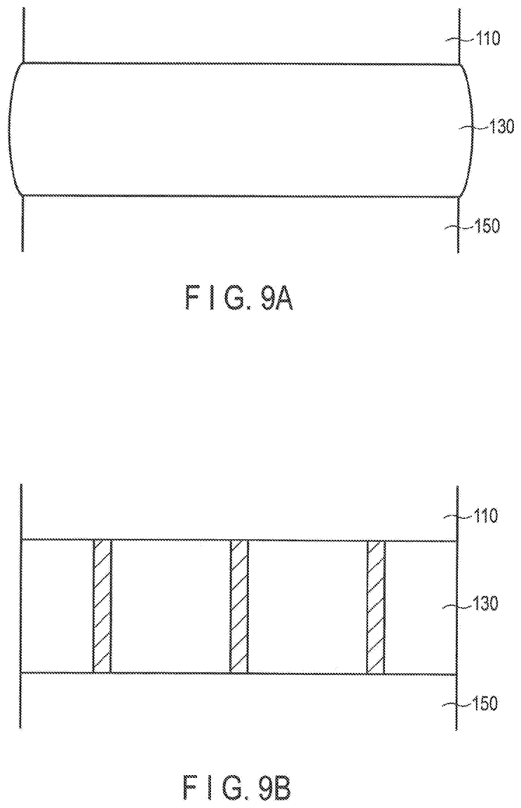

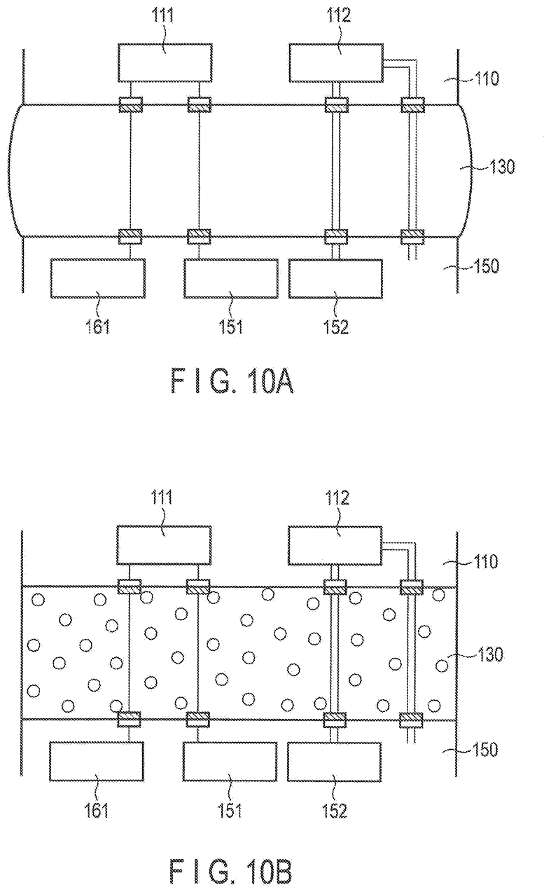

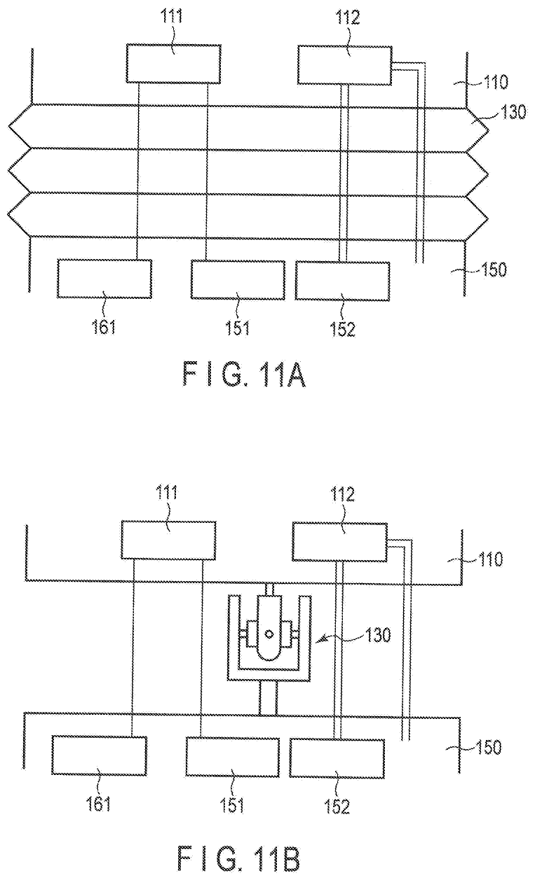

Next, the connecting unit 130 included in the blood pressure measuring apparatus 100 according to the present embodiment will be explained with reference to FIG. 7A, FIG. 7B, FIG. 7C, FIG. 8A, PIG. 8B, FIG. 9A, FIG. 9B, FIG. 10A, FIG. 10B, FIG. 11A and FIG. 11B.

FIG. 7A and FIG. 7B both shew a case in which the connecting unit 130 is made only of an impact absorbing material. In FIG. 7A, the connecting unit 130 is made of a sponge having good impact absorbability. In FIG. 7B, one or more (in this case, six) columnar solid bodies made of a material having good impact absorbability connect, the pulse wave detector 110 and the blood pressure measuring device 150 as the connecting unit 130. The material having good impact absorbability is, for example, a material that absorbs energy with almost no repulsion to an external force. However, since the connecting unit 130 only needs to connect the pulse wave detector 110 and the blood pressure measuring device 150 which are separated from each other, it does not have to be a material that particularly excels in impact absorbability.

In FIG. 7C, two columnar solid bodies connect the pulse wave detector 110 and the blood pressure measuring device 150 as the connecting unit 130. One of the solid bodies includes a signal line for transmitting an electrical signal, and connects the pressure pulse wave sensor 111 and the pulse wave measuring unit 151, and the electric power source unit 161, and the other solid body includes & duct that is a tube for carrying a fluid (for example, a gas), and connects a pressing unit 112 and the pump-and-valve 152, and the pressure sensor 153, etc.

The connecting unit 130 in FIG. 7A is, for example, a sponge made of polyurethane, which is a synthetic resin, a rubber material, or a foaming agent. The sponge is a porous soft material having numerous fine pores therein, and it is possible to freely adjust the impact absorbability through blending the rubber material and the foaming agent. In the example of FIG. 7A, the signal line connecting the pressure pulse wave sensor 111 and the electric power source unit 161, and the signal line connecting the pressure pulse wave sensor 111 and the pulse wave measuring unit 151 are hollowed out through the sponge. Furthermore, in the example of FIG. 7A, a duct, which is a tube carrying a gas, that connects the pressing unit 112 and the pump-and-valve 152, and a duct connecting the pressing unit 112 and the pressure sensor 153 (not shown) are hollowed out through the sponge. Furthermore, the connecting unit 130 may be, for example, a low resilience soft foam (for example, a styrene elastomer crosslinked foam) which excels in energy absorbability, or a low resilience urethane foam.

The material of the connecting unit 130 in FIG. 7B is, for example, a rubber material. A part of the columnar solid body that constitutes the connecting unit 130 includes a signal line, and another part of the columnar solid body includes a duct which is a tube that carries a gas. In the example of FIG. 7B, there are two signal lines, one connecting the pressure pulse wave sensor 111 and the electric power source unit 161, and the other connecting the pressure pulse wave sensor 111 and the pulse wave measuring unit 151, and two ducts, one connecting the pressing unit 112 and the pump-and-valve 152, and the other connecting the pressing unit 112 and the pressure sensor 153 (not shown).

The connecting unit 130 of FIG. 7C is, for example, made of the same material as the connecting unit 130 of FIG. 7B, which is a material that excels in impact absorbability. One of the columnar solid bodies constituting the connecting unit 130 includes a signal line, and the other one includes a duct which is a tube for conveying a fluid. In the example of FIG. 7C, there are two signal lines, one connecting the pressure pulse wave sensor 111 and the electric power source unit 161, and the other connecting the pressure pulse wave sensor 111 and the pulse wave measuring unit 151, and two ducts, one connecting the pressing unit 112 and the pump-and-valve 152, and the other connecting the pressing unit 112 and the pressure sensor 153 (not shown). Here, the connecting unit 130 is characterized by its structure and not the material.

For example, if the connecting unit 130 is a material that excels in impact absorbability, when the cuff of the blood pressure measuring device 150 is expanded or contracted, the movement of the blood pressure measuring device 150 is absorbed by the connecting unit 130, and will be difficult to be transmitted to the pulse wave detector 110. As a result, the accuracy of the pulse wave measurement of the pulse wave detector 110 will improve, and the blood pressure value for each pulse can be accurately measured. Furthermore, as shown in FIG. 7A and FIG. 7B, by including the signal line and the duct in the connecting unit 130, the electric power source unit 161, the pulse wave measuring unit 151, the pump-and-valve 152, and the pressure sensor 153 can be installed in the blood pressure measuring device 150 without being installed in the pulse wave detector 110. Therefore, the pulse wave detector 110 becomes compact and lightweight, the pressure pulse wave sensor 111 can be easily disposed on the radial artery, and the pressure pulse wave sensor 111 can reliably acquire the pulse wave. As a result, the accuracy of the pulse wave measurement of the pulse wave detector 110 will improve, and the blood pressure value for each pulse can be accurately measured.

FIG. 8A shows an example of a state in which two connecting lines connecting the pressure pulse wave sensor 111 and the electric power source unit 161, and the pulse wave measuring unit 151 exit the device of the pulse wave detector 110 and enter the blood pressure measuring device 150.

In FIG. 8A, it is assumed that the palm of the left hand is placed above. Therefore, the two connecting lines connecting the pressure pulse wave sensor 111 and the electric power source unit 161, and the pulse wave measuring unit 151 are all disposed outside the apparatus on the side of the arm on the thumb side of the left hand. By arranging the connecting lines in this manner, the connecting lines would net need to be disposed inside the connecting unit 130, which allows the width of the connecting unit 130 (the distance between the pulse wave detector 110 and the blood pressure measuring device 150) to be reduced, and allows the blood pressure measuring apparatus 100 to be made compact. Furthermore, the wires would be arranged outside, the apparatus on the side of the arm on the thumb side. Arms more easily interfere with surrounding objects on the outer arm side, i.e., on the little finger side, rather than the inner arm side, on the thumb side. Therefore, by arranging the wires on the side of the arm on the thumb side (the inner arm), the trouble of disconnection, etc. caused by the wires interfering with the surrounding objects is less likely to occur.

FIG. 8A shows an example of the blood pressure measuring apparatus 100 being attached to the left hand; however, the same also applies in the case where it is attached to the right hand. That is, also in the case of the right hand, the connecting line (electrically) connecting the pulse wave detector 110 and the blood pressure measuring device 150 would be arranged outside the apparatus on the side of the arm on the thumb side. The effect is similar to that in the case of the left hand.

FIG. 8B shows a case in which the connecting unit 130 existing between the pulse wave detector 110 and the blood pressure measuring device 150 in FIG. 8A is removed, and the duct connecting the pressing unit 112 and the pump-and-valve 152 etc. also exits the pulse, wave detector 110 and enters the blood pressure measuring device 150. That is, the blood pressure measuring apparatus 100 is in the extending direction of the arm to which it is attached, and does not have the connecting unit disposed between the pulse wave detector 110 and the blood pressure measuring device 150 (for example, a void is provided to form a gap). The connecting unit 130 includes a signal line and a duct, extends in a direction intersecting the extending direction of the arm (for example, passes outside the pulse wave detector 110 and the blood pressure measuring device 150), and connects the pulse wave detector 110 and the blood pressure measuring device 150.

In FIG. 8B, in the same manner as FIG. 8A, it is assumed that the palm of the left hand is upward. Therefore, all of the connecting units 130 including the signal line and the duct are disposed outside the apparatus on the side of the arm on the thumb side of the left hand. By arranging the connecting unit 130 in this manner, it is unnecessary to arrange the connecting unit 130 between the pulse wave detector 110 and the blood pressure measuring device 150 and on the extending direction of the arm, and thus the distance between the pulse wave detector 110 and the blood pressure measuring device 150 can be reduced, and the blood pressure measuring apparatus 100 can be made compact. Thus, the signal line and the duct are disposed outside the apparatus on the side of the arm on the thumb side. Arms more easily interfere with surrounding objects on the outer arm side, i.e., on the little finger side, rather than on the inner arm side, i.e., on the thumb side. Therefore, by arranging the signal line and the duct on the side of the arm on the thumb side (inner arm), the trouble of disconnection, etc. caused by the signal line and the duct interfering with surrounding objects is less likely to occur.

In addition, since a gap can be provided between the pulse wave detector 110 and the blood pressure measuring device 150, the arrangement of the pulse wave detector 110 and the blood pressure measuring device 150 can be easily and finely adjusted. As a result, since it becomes easy to arrange the pulse wave detector 110 and the blood pressure measuring device 150 at a desired position, the pulse wave detector 110 can acquire pulse waves with certainty, and the blood pressure measuring device 150 can measure biological information with high accuracy.

FIG. 8B shows an example of the blood pressure measuring apparatus 100 being attached to the left hand; however, the same also applies in the case where it is attached to the right hand. That is, the connecting unit 130 connecting the pulse wave detector 110 and the blood pressure measuring device 150 would be disposed outside the apparatus on the side of the arm on the thumb side also in the case of the right hand. The effect is similar to that in the case of the left hand.

FIG. 9A and FIG. 9B show the case in which both connecting units 130 contain gas. In FIG. 9A, the connecting unit 130 is a bag-like container containing gas. The container should toe made of a material that is flexible or elastic and does not leak gas, such as a rubber material Other materials for the container include vinyl chloride and silicone. In FIG. 9A and FIG. 9B, the signal line and duct, the inside of the pulse wave detector 110, and the inside of the blood pressure measuring device 150 are omitted.

The connecting unit 130 of FIG. 9A is, for example, a container of a rubber material that contains air, and has elasticity by adjusting the internal pressure of the bag so as to absorb a possible impact. The gas contained in this bag may be only a rare gas or nitrogen gas which has low chemical reactivity. The signal line and duct connecting the pulse wave detector 110 and the blood pressure measuring device 150 pass through the inside of the connecting unit 130, and the positions thereof are not particularly specified.

In the connecting unit 130 in FIG. 9B, one or more columnar solid bodies made of a material having good impact absorption is connected, to the pulse wave detector 110 and the blood pressure measuring device 150 in the same container as in FIG. 9A. As in the case of FIG. 7B, signal lines or ducts may pass through the interior of this solid body, or may be arranged in a space inside the container where solid bodies are not present, and gas is present.

If the connecting unit 130 is made of a material having elasticity by adjusting the internal pressure of the bag so as to absorb an impact, for example, when the cuff of the blood pressure measuring device 150 is inflated or contracted, the movement of the blood pressure measuring device 150 is absorbed by the connecting unit 130, and would be difficult to be transmitted to the pulse wave detector 110. As a result, the accuracy of the pulse wave measurement of the pulse wave detector 110 will improve, and the blood pressure value for each pulse can be accurately measured.

FIG. 10A and FIG. 10B show that both connecting units 130 have a detachable connector, and connect the pulse wave detector 110 and the blood pressure measuring device 150 by the connector. In either connecting unit 130 in FIG. 10A and FIG. 10B, some connectors also serve as an electric connection terminal to connect to a signal line, and electrically connect the pulse wave detector 110 and the blood pressure measuring device 150. Furthermore, in either connecting unit 130 of FIG. 10A and FIG. 10B, some connectors serve as a duct terminal to connect to a duct, and connect the pulse wave detector 110 and the blood pressure measuring device 150 by the duct. The surface of the connecting unit 130 in contact with the pulse wave detector 110 and the surface of the connecting unit 130 in contact with the blood pressure measuring device 150, for example, have a small protrusion on the surface, and cushion the impact between the pulse wave detector 110 and the blood pressure measuring device 150 and the connecting unit 130. Furthermore, since the projections are made of a material having a large coefficient of friction (for example, rubber), the pulse wave detector 110 and the blood pressure measuring device 150 and the connecting unit 130 do not easily shift. The signal line in the connecting unit 130 may be in the form of a film having excellent flexibility, and connectors may be connected to both ends.

The connecting unit 130 in FIG. 10A is a modification of the connecting unit 130 in FIG. 9A, in which connectors are provided on both sides of the connecting unit 130 of FIG. 9A, on the pulse wave detector 110 side and the blood pressure measuring device 150 side.

The connecting unit 130 in FIG. 10B is a modification of the connecting unit 130 in FIG. 7A, in which connectors are provided on both sides of the connecting unit 130 of FIG. 7A, on the pulse wave detector 110 side and the blood pressure measuring device 150 side, and the connecting unit 130 is made of a material having good impact absorption (for example, a rubber material).

The connectors of FIG. 10A and FIG. 10B are both electric connection terminals or duct terminals; however, the connectors are not limited thereto, and may merely connect the pulse wave detector 110 or the blood pressure measuring device 150 and the connecting unit 130 without serving as electrical connection terminals or duct terminals.

By setting the connector in this manner, it is possible to separate the pulse wave detector 110 and the blood pressure measuring device 150. Therefore, when one of the devices fails, only the failed device needs to be replaced. Therefore, since only the failed device needs to be replaced, the convenience for the user is enhanced.

In FIG. 11A, the connecting unit 130 has a bellows structure, and connects the pulse wave detector 110 and the blood pressure measuring device 150. FIG. 11B shows the pulse wave detector 110 and the blood pressure measuring device 15G connected by a universal joint.

By forming the connecting unit 130 in a bellows structure as shown in FIG. 11A, a closed space having a variable volume can be created, and by making the closed space airtight, it can serve as a resilient cushion. Therefore, the vibration of the blood pressure measuring device 150 is less likely to be transmitted to the pulse wave detector 110, and the pulse wave detector 110 can detect the pulse wave with high accuracy. In addition, the bellows structure allows the position of the pulse wave detector 110 and the blood pressure measuring device 150 located at both ends of the connecting unit 130 to be freely positioned not only in the expansion and contraction direction but also in a direction perpendicular thereto. Therefore, there is an effect that the pulse wave detector 110 and the blood pressure measuring device 150 may be freely arranged.

By connecting the pulse wave detector 110 and the blood pressure measuring device 150 by a universal joint as shown in FIG. 11B, the arrangement of the pulse wave detector 110 and the blood pressure measuring device 150 can be freely changed, and the pulse wave detector 11C and the blood pressure measuring device 150 would be less likely to interfere with each other. Therefore, the measurement accuracy of the pulse wave detector 110 and the blood pressure measuring device 150 is improved.

In the above-described embodiment, the pressure pulse wave sensor 111 detects, for example, a pressure pulse wave of the radial artery passing through a to-be-measured site (for example, the left wrist) (tonometry method). However, the present invention is not limited thereto. The pressure pulse wave sensor 111 may detect a pulse wave of a radial artery passing through a to-be-measured site (for example, the left wrist) as a change in impedance (impedance method). The pressure pulse wave sensor 111 may include a light-emitting element that emits light toward an artery passing through the corresponding portion of the to-be-measured site, and a light-receiving element that receives reflected light (or transmitted light) of the emitted light, and may be configured to detect the pulse wave of the artery as changes in volume (photoelectric method). The pressure pulse wave sensor 111 may also include a piezoelectric sensor in contact with the to-be-measured site, and may be configured to detect a strain caused by the pressure of the artery passing through the corresponding portion of the to-be-measured site as a change in electric resistance (piezoelectric method). Furthermore, the pressure pulse wave sensor 111 may include a transmitting element that transmits radio waves (transmission waves) toward an artery passing through the corresponding portion of the to-be-measured site, and a receiving element that receives reflected waves of the transmitted radio waves, and may be configured to detect a change in distance between the artery and the sensor caused by the pulse wave of the artery as a phase shift between the transmission waves and the reflected waves (radio wave irradiation method). As long as the method provides an observation of a physical quantity used to calculate the blood pressure, any method may be adopted in addition to the above.

In the embodiment described above, the blood pressure measuring apparatus 100 is assumed to be attached to the left wrist as a site to be measured. However, the present invention is not limited thereto, and the blood pressure measuring apparatus 100 may also be attached to the right wrist. The site to be measured should be where the artery passes, therefore, may be the upper limb such as the upper arm other than the wrist, or the lower limb such as the ankle or thigh.

According to the embodiment described above, since the pulse wave detector 110 that detects a pulse wave in a temporally continuous manner and the blood pressure measuring device 150 that measures biological information (first biological, information) intermittently are physically connected to be integrated with each other, the biological information measuring apparatus is made compact. Therefore, measuring can be facilitated, which enhances the convenience for a user. Furthermore, since a pulse wave is calibrated based on the biological information, the biological information (second biological information) is calculated from the pulse wave, and the pulse wave is calibrated based on the biological information measured by the blood pressure measuring device 150, it possible to calculate biological information with high accuracy from the pulse wave. Therefore, a user can easily obtain biological information with high accuracy. Furthermore, since the blood pressure measuring device 150 only performs intermittent measurement, the time during which the blood pressure measuring device 150 interferes with the user is reduced.

Furthermore, since the pulse wave detector 110 is disposed on the wrist of the living body, and the blood pressure measuring device 150 is disposed on the upper arm side of the pulse wave detector 110, the pulse wave can be reliably detected from the wrist. The length of the pulse wave detector 110 has a smaller width than the length of the blood pressure measuring device 150 in the arm-extending direction. This enables the blood pressure measuring device 150 to be arranged even closer to the palm. Thus, the pulse wave can be easily detected and the measurement accuracy can be maintained in a good condition. The pulse wave detector 110 differs in height between a first portion to be arranged on the palm side and a second portion to be arranged on the back side of the hand. The blood pressure measuring device 150 differs in height between a third portion to be arranged on the palm side and a fourth portion to be arranged on the back side of the hand. The first portion and the third portion differ in height. The second portion and the third portion differ in height. This makes it easy for a user to determine positions of the pulse wave detector 110 and the blood pressure measuring device 150 visually and haptically, thereby facilitating the positioning between the pulse wave detector 110 and the blood pressure measuring device 150.

The pulse wave detector 110 differs from the blood pressure measuring device 150 in terms of height from the surface of the arm at any position of the arm to which they are disposed. This makes it easy for a user to determine a position of the pulse wave detector 110 visually and haptically, thereby facilitating the positioning of the pressure pulse wave sensor 111. By measuring biological information with higher accuracy than that obtained from the pulse wave detector 110, and calibrating the biological information with high accuracy obtained from the blood pressure measuring device 150, the accuracy of biological information obtained based on a pulse wave from the pulse wave detector 110 can be ensured. This allows biological information to be calculated with high accuracy in a temporally continuous manner. Since the pulse wave detector 110 detects a pulse wave for each pulse, and the biological information relates to blood pressure, the biological information measuring apparatus is able to measure the blood pressure for each pulse wave per pulse in a temporally continuous manner. With the biological information measuring apparatus being constantly worn, accurate information can be acquired while calibrating biological information in a temporally continuous manner.

The apparatus of the present invention can also be realized by a computer and a program, and the program can be recorded on a recording medium or provided through a network.

Furthermore, each of the above-described devices and their device portions can be implemented either as a hardware configuration or as a combined configuration of hardware resources and software. The software of the combined configuration may be a program pre-installed in a computer from a network or a computer-readable storage medium to be executed by the processor of the computer for implementation of the functions of the respective devices.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

A part or all of the above-mentioned embodiments may also be described as in the following additional notes, without, limitation thereto.

(Additional Note 1)

A biological information measuring apparatus including a portion having impact absorbability that physically connects and integrates a portion for detecting a pulse wave and a portion for measuring biological information, and comprising a hardware processor and a memory,

wherein the hardware processor is configured to

detect the pulse wave in a temporally continuous manner,

intermittently measure first biological information, and

calibrate the pulse wave according to the biological information, and

the memory is

a memory device for storing the biological information.

(Additional Note 2)

A biological information measuring method in an apparatus including a portion having impact absorbability that physically connects and integrates a portion for detecting a pulse wave and a portion for measuring biological information, comprising:

detecting the pulse wave in a temporally continuous manner by using at least one hardware processor,

intermittently measuring the biological information by using at least one hardware processor, and

calibrating the pulse wave with the biological information by using at least one hardware processor.

(Additional Note 3)

A biological information measuring apparatus including a portion that physically connects a portion for detecting a pulse wave and a portion for measuring biological information, and comprising a hardware processor and a memory,

wherein the hardware processor is configured to

detect the pulse wave in a temporally continuous manner,

intermittently measure the biological information, and

calibrate the pulse wave according to the biological information, and

the memory is

a memory device for storing biological information calculated from the pulse wave.

(Additional Note 4)

A biological information measuring method in an apparatus including a portion that physically connects a portion for detecting a pulse wave and a portion for measuring biological information, comprising:

detecting the pulse wave in a temporally continuous manner by using at least one hardware processor,

intermittently measuring the biological information by using at least one hardware processor, and

calibrating the pulse wave with the biological information by using at least one hardware processor.

What is claimed is;

1. A biological information measuring apparatus comprising:
a detector configured to detect a pulse wave in a temporally continuous manner;
a measuring device configured to intermittently measure biological information and calibrate the pulse wave with the biological information; and
a connector having impact absorbability configured to physically connect and integrate the detector and the measuring device.

2. A biological information measuring apparatus comprising:
a detector configured to detect a pulse wave in a temporally continuous manner;
a measuring device configured to intermittently measure biological information and calibrate the pulse wave with the biological information; and
a connector configured to physically connect and integrate the detector and the measuring device.

3. The apparatus according to claim 1, wherein the detector is configured to have a volume and a mass smaller than those of the measuring device.

4. The apparatus according to claim 1, further comprising:
a drive unit configured to drive a pressing unit included in the detector and a cuff included in the measuring device; and
an electric power source unit configured to supply power to units included in the detector and the measuring device,
wherein the drive unit and the electric power source unit are configured to be included in the measuring device.

5. The apparatus according to claim 4, wherein the drive unit comprises a pump-and-valve and a pressure sensor, and adjusts a pressure of the cuff or the pressing unit.

6. The apparatus according to claim 1, further comprising:
a first drive unit configured to drive a pressing unit included in the detector;
a second drive unit configured to drive a cuff included in the measuring device; and
an electric power source unit configured to supply power to units included in the detector and the measuring device,
wherein the first drive unit is configured to be included in the detector, and the second drive unit and the electric power source unit are configured to be included in the measuring device.

7. The apparatus according to claim 6, wherein the first drive unit and the second drive unit respectively comprise a pump-and-valve and a pressure sensor, and adjust a pressure of the cuff or the pressing unit.

8. The apparatus according to claim 1, further comprising a display configured to display a detection result of the detector or a measurement result of the measuring device, wherein the display is included in the measuring device.

9. The apparatus according to claim 1, further comprising a first display configured to display a detection result of the detector, and a second display configured to display a measurement result of the measuring device, wherein the first display is included in the detector, and the second display is included in the measuring device.

10. The apparatus according to claim 1, further comprising an operation unit configured to operate the detector and the measuring device, wherein the operation unit is included in the measuring device.

11. The apparatus according to claim 1, further comprising a first operation unit configured to operate the detector, and a second operation unit configured to operate the measuring device, wherein the first operation unit is included In the detector, and the second operation unit is included in the measuring device.

12. The apparatus according to claim 1, wherein the connecting unit is configured to extend in a direction connecting the detector and the measuring device in a straight line, and connect the detector and the measuring device.

13. The apparatus according to claim 1, wherein the connecting unit is configured to extend in a direction intersecting with a direction connecting the detector and the measuring device in a straight line to connect the detector and the measuring device.

14. The apparatus according to claim 1, wherein the detector and the measuring device are configured to be installed on a wrist, and the connecting unit is configured to extend from the detector and the measuring device in a direction intersecting a direction in which an arm is extended to connect the detector and the measuring device.

15. The apparatus according to claim 1, wherein the connecting unit is configured to connect the detector and the measuring device with a detachable connector.

16. The apparatus according to claim 15, wherein a part of the connector is configured to be connected to a signal line for transmitting an electrical signal between the detector and the measuring device, and
in a case where the drive unit is included only in the measuring device, another part of the connector is configured to be connected to a tube through which gas flows in and out between the detector and the measuring device.

17. The apparatus according to claim 1, wherein the connecting unit is configured to connect the detector and the measuring device by a tube having a bellows structure.

18. The apparatus according to claim 1, wherein the connecting unit is configured to connect the detector and the measuring device by a universal joint.

19. The apparatus according to claim 1, wherein the measuring device is configured to measure the biological information with higher accuracy than biological information obtained from the detector.

20. The apparatus according to claim 1, wherein the detector is configured to detect the pulse wave per pulse, and the biological information is blood pressure.

21. A biological information measuring method in a biological information measuring apparatus comprising a connecting unit having impact absorbability, which physically connects and integrates a detector that detects a pulse wave and a measuring device that measures biological information, the method comprising:
detecting the pulse wave in a temporally continuous manner;
intermittently measuring the biological information; and
calibrating the pulse wave according to the biological information.

22. A non-transitory computer readable medium storing a computer program which is executed by a computer to provide the steps of, the computer being included in the apparatus that comprises a connecting unit having impact absorbability, which physically connects and integrates a detector that detects a pulse wave and a measuring device that measures biological information:
detecting a pulse wave in a temporally continuous manner;
intermittently measuring biological information; and
calibrating the pulse wave according to the biological information.
